(19) United States
(12) Patent Application Publication
(10) Pub. No.: US 2023/0149208 A1
(43) Pub. Date: May 18, 2023

(54) NON-INVASIVE CONTRACEPTIVE DEVICE

(71) Applicant: Malgorzata Maria Serafin-Fahimi, Franklin Lakes, NJ (US)

(72) Inventor: Malgorzata Maria Serafin-Fahimi, Franklin Lakes, NJ (US)

(21) Appl. No.: 17/528,278

(22) Filed: Nov. 17, 2021

(51) Int. Cl. A61F 6/22; A61F 6/18

(52) U.S. Cl. CPC A61F 6/225; A61F 6/18

(57) ABSTRACT

A device for occluding a tubular body lumen that includes a structure having a proximal and a distal end and having a means for securing said device to the wall of a fallopian tube prior to tissue in-growth; the device for occluding a tubular body lumen wherein the device is constructed from a radio-opaque substance; the distal end of the occlusion device includes a connection means for connecting to an insertion rod; wherein said device has a plurality of spikes, bumps, grooves, protrusions to secure said device to the wall of a fallopian tube prior to tissue in-growth and removal of an insertion rod; wherein said device having one of a rigid outer or semirigid outer construction; The occluding device having one of a smooth, a porous, and a combination smooth and porous surface; wherein the occluding device includes antimicrobial properties; wherein the proximal and distal ends of the device comprise a circular shape, an oval shape, a half oval shape, a cylindrical shape, a cone shape, a bullet shape, a sphere shape or a combination thereof; and wherein the occluding device is one of a solid, hollow, and a combination of solid and hollow.

### FIELD OF THE INVENTION

The present invention relates to the field of contraceptive devices. More particularly, the invention relates to a female contraceptive device that is implanted via a non-invasive occlusion of both fallopian tubes.

### BACKGROUND

Female contraception and/or sterilization may be affected by transcervically in introducing an object bilaterally into fallopian tubes to inhibit conception. Devices, systems, and methods for such a contraceptive approach have been described in various patents and patent applications. For example, U.S. Pat. Nos. 6,526,979 6,634,361 describe devices that are transcervically inserted via an ostium of a fallopian tube and mechanically anchored within the fallopian tube. One example of such a device is known as “Essure” from Conceptus, Inc. of Mountain View, Calif.

Tissue in-growth tends to provide a long-term contraception and/or permanent sterilization. Tissue in-growth is not immediate, and, as in the case of Essure noted above, physicians typically advised patients to wait to have unprotected intercourse for about three months after insertion of the device for long-term contraception/sterilization based upon tissue in-growth to become effective.

Prior art occlusion devices have several problems associated with their use. For example, some of the problems with their use include tear or hole (i.e*., “perforation”) of the uterus and/or the fallopian tube (s). Other problems include the implanted device may move unexpectantly to the abdominal or pelvic cavity causing damage to the fallopian tube and such lack of closure of the tube (due to an incomplete or disrupted tissue in-growth process) may cause unintended pregnancy and infection.

Accordingly it would be desirable to provide an occlusion device that assists in preventing perforations of the uterus and fallopian tubes or from falling back into the uterine cavity.

It would also be desirable to provide an occlusion device to prevent unintended pregnancy.

It would be further desirable to provide an occlusion device that resists movement of the occlusion device into the abdominal or pelvic cavity.

It would be still further desirable to provide an occlusion device that resists post-operative infections.

It would be yet still further desirable to provide an occlusion device that has deployable securing means to secure the occlusion device to the wall of the fallopian tube.

Accordingly, it is an object of the present invention to provide an occlusion device that assists in preventing perforations of the uterus and fallopian tubes.

It is another object of the present invention is to provide an occlusion device to prevent unintended pregnancy.

It is a further object of the present invention to provide an occlusion device that resists movement into the abdominal or pelvic cavity.

It is still a further object of the present invention to provide an occlusion device that resists post-operative infection.

### SUMMARY OF THE INVENTION

Various embodiments of the present invention are disclosed below, and the following summary provides a brief description of only some of these embodiments.

In an embodiment, the present invention relates to a device for occluding a tubular body lumen that may include a device having a proximal end (meaning front/top) and a distal end (meaning back/bottom). The occlusion device may include a means for anchoring the occlusion device to the wall of a fallopian tube prior to tissue in-growth. The occlusion device may have fixed anchor points or deployable anchor points. The occlusion device may be constructed partially or fully from a radio-opaque substance to determine its proper location within the fallopian tube, so that is viewable on an x-ray or other diagnostic equipment. The occlusion device may have two or more spikes, bumps, grooves, protrusions on the surface of the occlusion device to secure the device to the wall of a fallopian tube prior to tissue in-growth and assist with the removal of an insertion rod by using the anchoring means to provide resistance against the force being applied to remove the insertion rod. Furthermore, the porous surface is to improve and enhance the process of tissue in-growth into the occlusion device's outer wall. The distal end or back/bottom of the occlusion device includes a connection means for connecting to the insertion rod. The connection means may include one or more or the following structures, for example, a groove, a suction dimple, a twist lock/unlock, or any other suitable connection means that allows the occlusion device to be temporarily secured to the insertion rod. Other aspects or the present invention may further include the outer construction of the occlusion device being constructed from a rigid or semirigid material. Further, other aspects of the present invention may include a smooth, a porous, and a combination smooth and porous outer surface to promote tissue in-growth.

Still further aspects of the present invention may include an occlusion device that has antimicrobial properties coated on or impregnated with, such as, for example, silver nanoparticles to prevent post-operative infection.

Yet still further, the present invention may include the proximal and distal ends of the occlusion device having a circular shape, an oval shape, a half oval shape, a cylindrical shape, a cone shape, a cylinder with a rounded top (i.e., bullet shape), a sphere shape or any other shape that would provide obstruction of the fallopian tube and allow for tissue in-growth, which would cause semi-permanent or permanent sterilization. Additionally, the distal end of the occlusion device may include a means of inserting the occlusion device into the fallopian tube and deploying deployable anchors.

It is therefore an object of the present invention to provide a female contraception/serialization device that minimizes infection.

It is a further object of the present invention to provide a female contraception serialization device that promotes in-growth of tissue around the occlusion device such that upon implanting the device, the occlusion device begins working immediately due to the device being secured via a tight fit within the fallopian tube by a securing means, which may be a plurality of spikes, bumps, grooves, protrusions on the surface of the occlusion device. Also, the size of the occlusion device is a means to create a tight fit placement of the occlusion device within the tubular wall of the fallopian tube.

### BRIEF DESCRIPTION OF THE DRAWINGS

The present invention is described by way of example with reference to accompanying drawings, wherein:

FIG. 1 is a schematic view illustrating the uterine and tubal anatomy for deployment of the contraceptive device into the intra-mural portion of the fallopian tube.

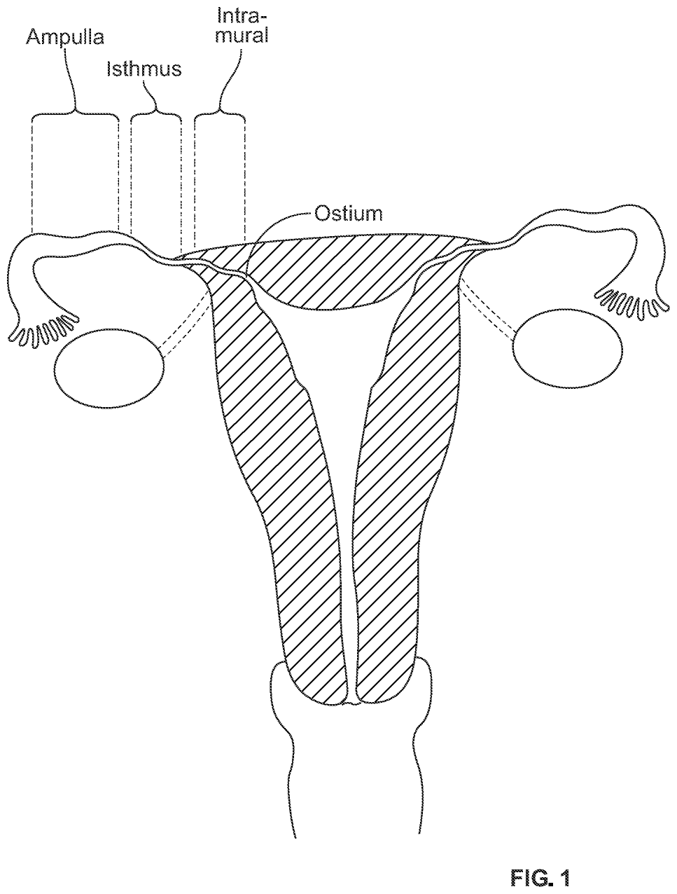

FIG. 2a-2d illustrate different views of one example of an occlusion device in accordance with embodiments of the present invention.

FIG. 3A-3E are illustrative examples of deployable and adjustable anchoring system and different shapes of the occlusion device according to embodiments of the present invention.

FIG. 4A is an illustrative example of an insertion means according to an embodiment of the present invention.

FIG. 4B is a cross sectional view of a tip of an insertion means showing how and where the insertion rod connects to the occlusion device and other aspects of the present invention.

FIG. 5 is an illustrative example of an hysteroscope that may be used with embodiments of the present invention.

### DETAILED DESCRIPTION

The present invention discloses a contraceptive device and insertion means that provides substantially immediate and permanent sterilization.

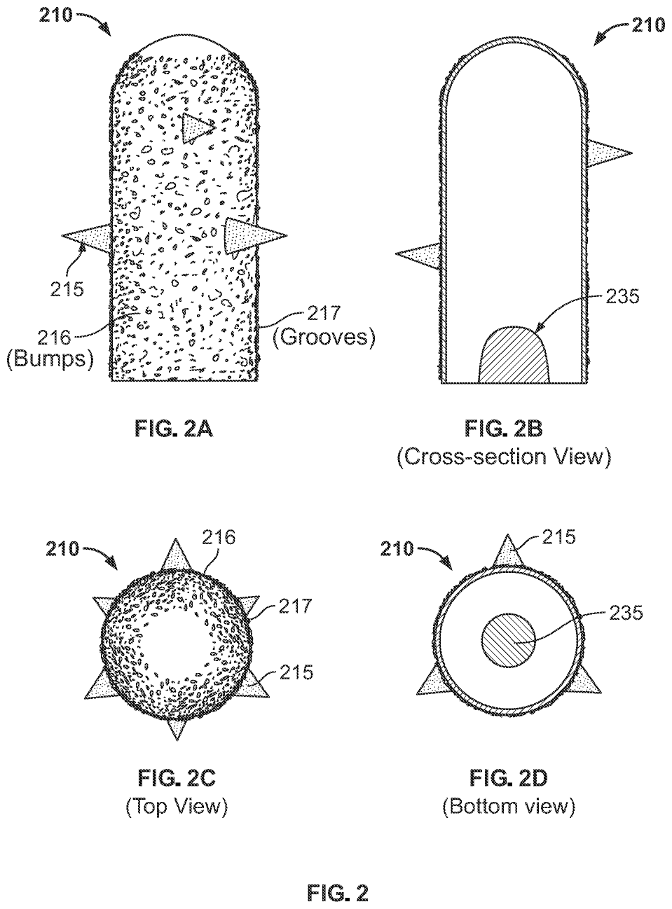

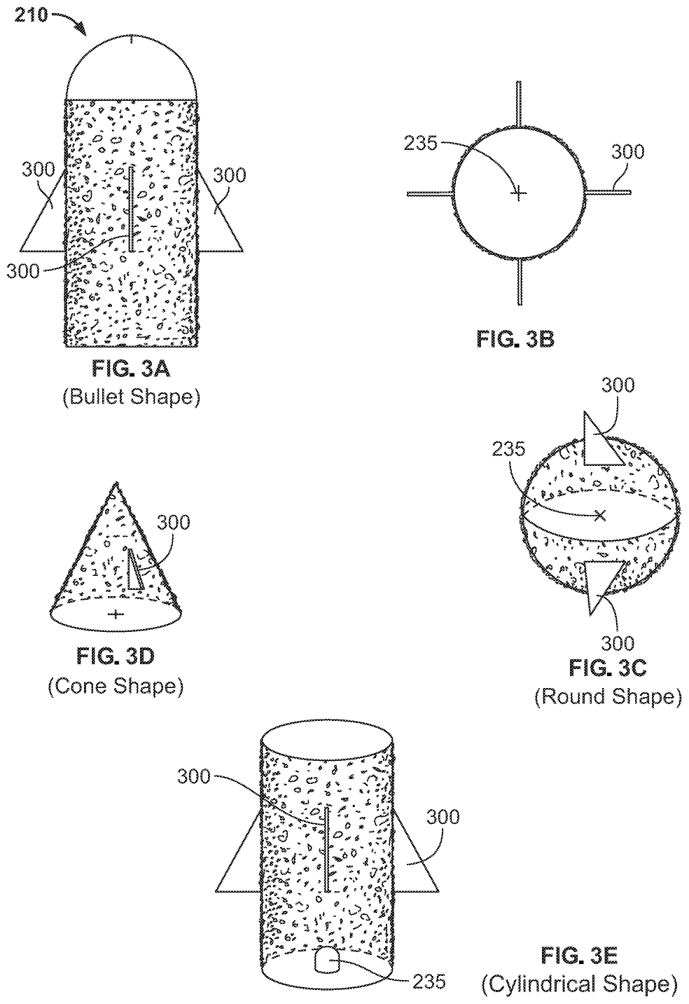

More particularly, the present invention may include an embodiment of an occlusion device having a proximal and a distal end. The occlusion device may include an anchoring means (215, 216, 217) shown in FIG.2 for securing the occlusion device to the wall of a fallopian tube prior to tissue in-growth. For example, the anchoring means for securing the occlusion device may include two or more fixed spikes, bumps, grooves, protrusions on the surface of the occlusion device and/or may include a deployable anchoring system 300 shown in FIG. 3 that may be used to secure the occlusion device to the fallopian tube wall.

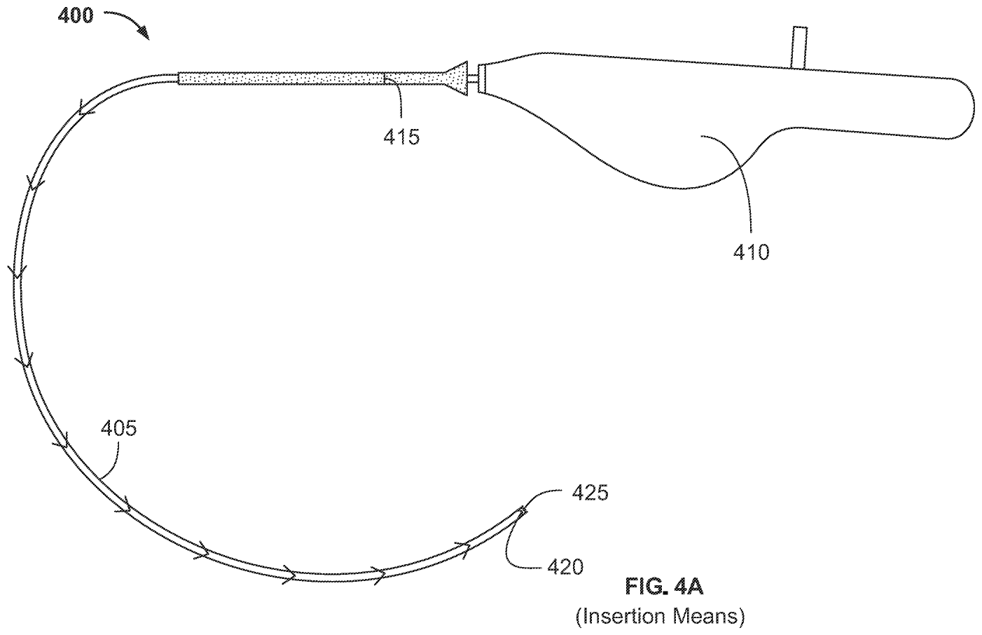

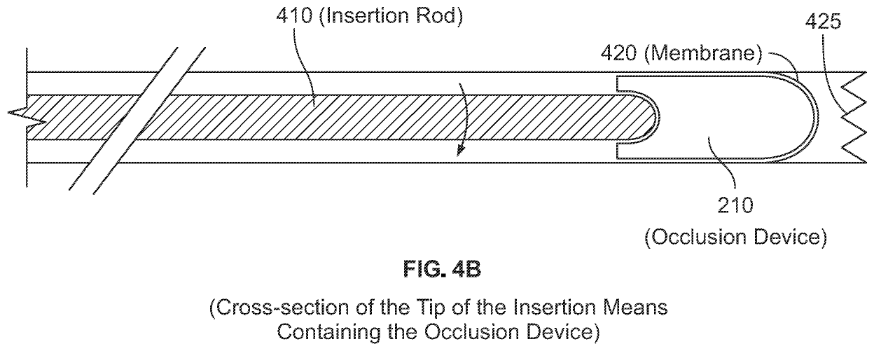

FIG. 2 shows illustrative examples of different views contemplated for occlusion device 210 and different non-deployable anchoring means 215, 216, 217 for securing occlusion device 210 to the fallopian tube wall. FIGS. 2A and 2B show a longitudinal view and a longitudinal cross-sectional view of occlusion device 210 as a bullet shape with two or more non-deployable anchoring means 215, 216, 217 on the surface of the occlusion device. FIG. 2C shows a top view with non-deployable anchoring means 215, 216, 217. FIG. 2D shows a bottom view of occlusion device 210 with connection means 235. In FIGS. 2B and 2D also shows one embodiment of a connection means 235 where insertion rod 410 may be connected to the occlusion device (shown in FIG. 4B) As set forth above, connection means may include a groove, a suction dimple, a twist lock/unlock, or any other suitable connection means that allows the occlusion device to be temporarily secured to insertion rod 410.

FIG. 3 shows illustrative examples of different shapes contemplated for an occlusion device. FIG. 3 also shows illustrative examples of a deployable anchoring means 300 in a deployed position. Occlusion device 210 may include two or more deployable anchor means 300. Deployable anchor means 300 may have smooth or jagged edges and/or may include any other subtable type of surface to promote tissue in-growth to secure the occlusion device to the fallopian tube wall. FIG. 3A shows an occlusion device with anchor means 300, which may be deployed from the surface or from within the body of the occlusion device by rotating insertion rod 410 using any suitable means for rotating the rod. For example, insertion rod 410 may be connected to a knob, a handle, a button/trigger, or any other device that when rotated or actuated causes the occlusion device to deploy the anchoring means. Anchor means 300 may be deployed in predetermined distances with each rotation of the rotation means (shown in FIG. 4B) (e.g., one micron per revolution or half a micron per revolution, etc.) allowing for a custom fit of the occlusion device. These custom fit deployable anchors are designed to prevent rupturing of the fallopian tube wall by allowing the anchoring means to deploy in miniscule increments to prevent movement and provide enough resistance to remove insertion rod 410.

All of the various occlusion device embodiments may be coated, impregnated, or made from an antimicrobial substance such as, for example, silver, tungsten carbide and copper nanoparticles. These antimicrobial substances may work alone or in combination with each other to prevent or reduce the possibilty of infection. One skilled in the art will appreciate that other antimicrobial substances may be used to prevent or impede infections and such use is explicitly contemplated herein.

Occlusion device 210 may be constructed partially or fully from a radiopaque substance (not shown). A radio radiopaque substance blocks radiation rather than allow it to pass through. In other words, the occlusion device would show up on an x-ray film in order to determine the desired position of an occlusion device 210 within the fallopian tube.

Other aspects of the present invention may further include the outer shell of the occlusion device being constructed from a rigid material, such as, for example, metal or semirigid material such as, for example, high density polyethylene (not shown). One skilled in the art will appreciate that other substances may be used and are specifically contemplated herein. Still other features and aspects of the present invention may further include smooth, a porous, and a combination smooth and porous outer surface.

FIG. 4A shows an illustrative example of an insertion means 400 with handle 410, delivery Catheter 405, protective membrane 420, introducer 415, and teeth-like structures 425 at the very end of delivery catheter 405, which may be needed for a secure and firm placement of the insertion catheter 405 against the uterine wall and encircling an ostium immediately prior to the deployment of an occlusion device 210 into a fallopian tube. Additionally these teeth-like structures 425 may be in a shape/form of spikes, needles and other types of protrusions. FIG. 4B is showing a cross-sectional view of a tip of delivery catheter 405 with membrane 420, insertion rod 410, occlusion device 210, and teeth-like structures 425. Insertion rod 410 may be used to insert occlusion device 210 into a fallopian tube. Membrane 420 may be a one-way penetrable membrane that keeps occlusion device 210 in place while passing through a working/operative channel of the hysteroscope. For example, in operation, catheter 405 may be used to house occlusion device 210. Membrane 420 in conjunction with insertion rod 410 holds occlusion device 210 in place prior to its insertion into a fallopian tube. Once at the desired location at the ostium, a force is applied to insertion rod 410, which advances occlusion device 210 forward and pushes through membrane 420 and into a fallopian tube.

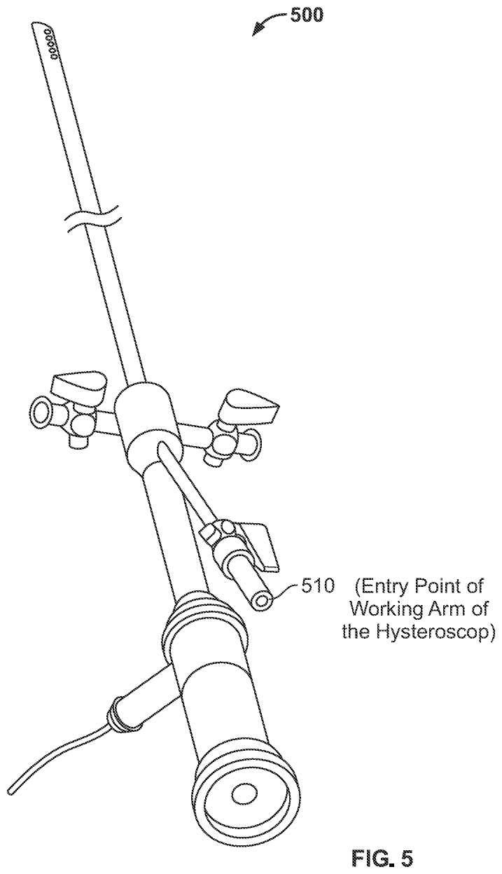

Further, FIG. 4A shown an illustrative embodiment of a delivery catheter 405, which may be a flexible catheter. Further, FIG. 4A shows introducer 415, which may start at the base of delivery catheter 405 and advance along catheter 405 and continues to advance until it covers and protects a tip of the delivery catheter with a preloaded occlusion device. Once a scope has been placed within the female introducer 215 with the distal portion of the catheter is passed through the working/operating channel of a scope 500 (shown in FIG. 5) into the uterine cavity.

FIG. 5 shows a prior art hysteroscope with its entry point 510 into the working/operating channel that may be used in connection with the present invention to assist in inserting of occlusion device 210 by providing a safe pathway via operating channel from the outside of the female's body into the uterine cavity.

The foregoing description with attached drawings is only illustrative of possible embodiments of the described devices and should only be construed as such. Other persons of ordinary skill in the art will realize that many other specific embodiments are possible that fall within the scope and spirit of the present idea. The scope of the invention is indicated by the following claims rather than by the foregoing description. Any and all modifications which come within the meaning and range of equivalency of the following claims are to be considered within their scope.

What is claimed is:

1. A device for occluding a tubular body lumen that includes a structure having a proximal and a distal end comprising:
a means for securing said device to the wall of a fallopian tube prior to tissue in-growth;
the device for occluding a tubular body lumen wherein said device being constructed from a radio-opaque substance, and wherein said device having a plurality of spikes, bumps, grooves, protrusions to secure said device to the wall of a fallopian tube prior to tissue in-growth and removal of an insertion rod;
the distal end of the occlusion device includes a connection means for connecting to the insertion rod.

2. The device as set forth in claim 1 having a rigid outer construction.

3. The device as set forth in claim 1 having a semirigid outer construction.

4. The device as set forth in claim 3 having one of a smooth, a porous, and a combination smooth and porous outer surface.

5. The device as set forth in claim 4, wherein the device includes antimicrobial properties.

6. The device as set forth in claim 5, wherein the device is coated with a substance having antimicrobial properties.

7. The device as set forth in claim 5, wherein the device is impregnated with a substance having antimicrobial properties.

8. The device as set forth in claim 5, wherein the substance having antimicrobial properties includes silver nanoparticles.

9. The device as set forth in claim 1, wherein the device has dimensions between 1 mm and 10 mm.

10. The device set forth in claim 9, wherein the proximal and distal ends of the device comprise a circular shape, an oval shape, a half oval shape, a cylindrical shape, a cone shape, a bullet shape, or a sphere shape.

11. The device as set forth in claim 9, wherein the occluding device is one of a solid, hollow, and a combination of solid and hollow.

12. The device set forth in claim 1, wherein the occluding device is pre-sealed within a delivery catheter capped with a penetrable membrane.

13. A device for occluding a tubular body lumen that includes a structure having a proximal and a distal end comprising:
a means for securing said device to the wall of a fallopian tube prior to tissue in-growth,
the device for occluding a tubular body lumen wherein said device is constructed from a radio-opaque substance;
the distal end of the occlusion device includes a connection means for connecting to the insertion rod;
wherein said device has a plurality of spikes, bumps, grooves, protrusions to secure said device to the wall of a fallopian tube prior to tissue in-growth and removal of an insertion rod;
wherein said device having one of a rigid outer or semirigid outer construction;
The occluding device having one of a smooth, a porous, and a combination smooth and porous surface; and
wherein the occluding device includes antimicrobial properties.

14. The device as set forth in claim 13, wherein the antimicrobial properties are impregnated with a substance having antimicrobial properties.

15. The device as set forth in claim 13, wherein the antimicrobial properties are coated with a substance having antimicrobial properties.

16. The device as set forth in claim 13, wherein the antimicrobial properties are coated and impregnated with a substance having antimicrobial properties.

17. The device as set forth in claim 13, wherein the substance having antimicrobial properties includes silver nanoparticles.

18. The device as set forth in claim 14, wherein the proximal and distal ends of the device comprise a circular shape, an oval shape, a half oval shape, a cylindrical shape, a cone shape, a bullet shape, or a sphere shape.

19. The device as set forth in claim 17, wherein the occluding device is one of a solid, hollow, and a combination of solid and hollow.

20. The device set forth in claim 13, wherein the occluding device is pre-sealed within an introducer capped with a penetrable membrane.

21. A device for occluding a tubular body lumen that includes a structure having a proximal and a distal end comprising:
a means for securing said device to the wall of a fallopian tube prior to tissue in-growth,
the device for occluding a tubular body lumen wherein said device is constructed from a radio-opaque substance;
the distal end of the occlusion device includes a connection means for connecting to ab insertion rod;
wherein said device has a plurality of spikes, bumps, grooves, protrusions to secure said device to the wall of a fallopian tube prior to tissue in-growth and removal of an insertion rod;
wherein said device having one of a rigid outer or semirigid outer construction;
The occluding device having one of a smooth, a porous, and a combination smooth and porous surface;
wherein the occluding device includes antimicrobial properties;
wherein the proximal and distal ends of the device comprise a circular shape, an oval shape, a half oval shape, a cylindrical shape, a cone shape, a bullet shape, a sphere shape or a combination thereof; and
wherein the occluding device is one of a solid, hollow, and a combination of solid and hollow.

22. The device set forth in claim 19, wherein the occluding device is pre-sealed within a delivery catheter capped with a penetrable membrane.
